(19) United States
(12) Patent Application Publication
(10) Pub. No.: US 2025/0269136 A1
(43) Pub. Date: Aug. 28, 2025

(54) NO supply installation to supply an emergency flow in the case of malfunctioning of the flow sensor

(71) Applicant: INOSYSTEMS, Antony (FR)

(72) Inventors: Frederic MARCHAL, Antony (FR); Mary SCHMITT, Antony (FR); Thierry BOULANGER, Antony (FR); Yann BLANDIN, Antony (FR)

(21) Appl. No.: 19/048,690

(22) Filed: Feb. 7, 2025

(30) Foreign Application Priority Data
Feb. 23, 2024 (FR) 2401784

(51) Int. Cl. A61M 16/12; A61M 16/00; A61M 16/10

(52) U.S. Cl. CPC A61M 16/12; A61M 16/1005; A61M 2016/0039; A61M 2202/0275; A61M 2205/505

(57) ABSTRACT

The invention concerns an installation (100) to supply a gaseous mixture containing NO comprising an NO supply apparatus (1) connected to a respiratory circuit (20; 21) with an injection device (24) and a flow sensor (25) in order to measure the flow of respiratory gas. The NO supply apparatus (1) comprises a gas circuit (200) with flow control means (220, 221) in order to control the flow of gas in the internal gas circuit (200), and control means (210) in order to determine a set gas flow containing NO, and to control the flow control means (220, 221); and storage means (212) in order to store successive set flows. In the case of interruption of transmission of the flow measurements carried out by the flow sensor (25), the control means (210) control the flow control means (220, 221) in order to supply the gas to an emergency flow calculated from stored set flows.

### TECHNICAL FIELD

The invention concerns an installation for supply of a gaseous mixture based on NO to a patient, typically an NO/nitrogen (N₂) mixture, comprising an NO supply apparatus which can supply an emergency flow of NO in the case of malfunctioning resulting in an interruption of transmission by the flow sensor to the means for controlling the NO supply apparatus, of any measurement of flow of respiratory gas based on oxygen, such as air, or an NO/N₂ mixture, coming from a medical ventilator, typically a respiratory gas coming from a medical ventilator, i.e. in the case of loss of the flow signal.

### BACKGROUND

Inhaled nitrogen monoxide (NO or NOi) is a gaseous medication commonly used to treat patients suffering from acute pulmonary arterial hypertension, in particular pulmonary vasconstrictions in adults or children, including newborn babies (PPHN), as described for example by EP-A-560928 or EP-A-1516639.

In order to implement therapy by inhaled NO, use is made of a gas supply installation, also known as an NO administration installation, comprising an NO supply apparatus and a medical ventilator, i.e. a respiratory assistance apparatus, supplying a patient circuit. The NO supply apparatus makes it possible to inject a gaseous mixture based on NO, typically an NO/nitrogen mixture, into the patient circuit, which is also supplied with a gaseous flow containing oxygen (at least approximately 20% by volume), such as air or an oxygen/nitrogen mixture (O₂/N₂), supplied by the medical ventilator. In general, the patient circuit comprises one or a plurality of flexible ducts which are connected fluidically to a respiratory interface, such as a tracheal intubation sensor or the like, which is used to supply to the patient to be treated a therapeutic gaseous mixture containing a given quantity or dose of NO, i.e. a posology, typically of between 5 and 40 ppmv of NO.

A gas supply installation of this type is described for example by EP3821929. This type of installation is used in a hospital environment in order to administer the treatment by NO, and thus care for patients who need to inhale NO in order to treat their pulmonary arterial hypertension.

Installations of this type are also described by EP4209243, EP4241817, EP4241812 and EP4295882.

In order to be able to supply to the patients a therapeutic gaseous mixture containing an NO content corresponding to the desired posology, the NO supply apparatus must comprise means or a system for controlling the flow which make it possible to control or adjust the flow of NO/N₂ supplied, for example a system including one or more proportional valves or the like controlled by means for controlling the apparatus.

However, the flow of NO/N₂ to be supplied depends in particular on the flow of respiratory gas (i.e. air or O₂/N₂) coming from the medical ventilator.

Thus, a flow sensor is habitually used which is arranged in the respiratory circuit between the medical ventilator and the site of injection of the NO/N₂ mixture, in order to carry out respiratory gas flow measurements (quasi-)continuously These flow measurements are then transmitted to the control means, which use them to calculate a set NO flow, which is used to control the flow control means, typically one or more proportional valves, or the like.

However, during the use of an NO supply installation, it may happen that the measurement of the respiratory gas flow by the flow sensor is disrupted, defective, or impossible to carry out, for example in the case of malfunctioning or accidental disconnection of the flow rate sensor, thus generating an interruption in the transmission of the flow measurements by the flow sensor.

This interruption of transmission of the flow measurements causes a major safety problem for the patient, since, in the absence of flow measurement, the control means can no longer calculate the set NO flow, which is used to control the flow control means, and thus the supply of NO is carried out erroneously or may even be interrupted.

In order to prevent this, the apparatus must be able to supply the NO to the patient at the required posology, by going into a so-called “emergency” or “emergency dosage” supply mode, in which the set NO flow is calculated even in the absence of a flow measurement, i.e. even in the case of interruption of the transmission of the flow measurements by the flow sensor to the control means.

Thus, EP3233171 proposes supplying a predetermined fixed flow of NO/N₂ mixture in the case of detection of an interruption of transmission of the flow measurements by the flow sensor. This is not precise, and leads to substantial fluctuations of the NO content of the combined gaseous mixture, since the flow of NO/N₂ is fixed, whereas that of the respiratory gas, in which the flow of NO/N₂ is injected in order to form the combined gaseous mixture, varies over a period of time.

In addition, EP3410927 proposes storing a history of values of flow measurements carried out by the flow sensor, and using this history of stored values in order to calculate the set NO/N₂ flow, in the case of interruption of transmission of the flow measurements. This solution is not ideal since the “rough” flow values stored can be erroneous, in particular in the case of intermittent malfunctioning of the flow sensor, which can for example be derived from untimely electrostatic discharges on the sensor, leading to storage of incorrect respiratory gas flow values. Since the flow values are erroneous, the flow calculations which use them are thus also erroneous, which results in an inadequate quantity of supply of NO/N_{2.}

In addition, EP3410927 proposes using an additional NO sensor in order to measure the flow of NO/N₂ supplied by the apparatus over a period of time, and storing a history of the NO flow measurements carried out by the additional NO sensor. In the case of interruption of transmission of the respiratory gas flow measurements by the respiratory gas sensor, the apparatus uses the history of the NO flow values stored in order to establish the set NO/N₂ flow. In this case also, the solution is not ideal, since it complicates the global architecture of the apparatus by requiring the incorporation of an additional sensor. In addition, working on historic values of NO previously supplied has disadvantages, since these flow values can be erroneous, for example because of an excessively high signal/noise ratio, in particular for the NO values supplied which are very low (i.e. ppmv).

A problem therefore consists of proposing an improved installation for supply of NO, which makes it possible to determine a set NO flow, in the absence of measurement of respiratory gas flow, in other words in the case of interruption of the transmission to the control means (i.e. loss of the respiratory flow signal), of the respiratory gas flow measurements carried out by the flow sensor, so as to be able to supply to the patient a final gaseous mixture based on NO containing a proportion of NO which is equal or close to a posology determined by a member of the medical staff, i.e. a doctor or the like.

### SUMMARY

A solution according to the invention concerns an installation for supply of a gaseous mixture containing NO, also known as an “NO supply installation” to a patient, comprising:
an NO supply apparatus which is supplied with a gas containing NO in a given initial proportion, typically a NO/N₂ mixture, and configured to supply the gas containing NO;
a respiratory circuit comprising an injection device which is configured to mix gas containing NO coming from the NO supply apparatus with a flow of respiratory gas containing O₂ conveyed by the respiratory circuit, and obtain a combined gaseous mixture containing NO and oxygen; and
a flow sensor which is configured to measure at least one respiratory gas flow within the respiratory circuit, and provide at least one respiratory gas flow measurement.

The NO supply apparatus of the NO supply installation comprises:
an internal gas circuit to convey the gas containing NO;
means for controlling the main and secondary flows which are configured to control (i.e. permit, prevent, prohibit, adjust etc.) the flow of gas containing NO conveyed by the internal gas circuit;
(micro)processor control means which are configured to:
   determine at least one set gas flow containing NO to be supplied to the injection device, from at least one respiratory gas flow measurement carried out by the flow sensor; and
   control at least part of the main and secondary flow means in order to supply the gas containing NO to the set flow determined;
and storage means.

In addition, in the installation for supply of NO (i.e. nitrogen monoxide), such as an NO/N₂ mixture:
the storage means are configured to store successive set flows of gas containing NO which have been determined by the control means; and
in the case of interruption of transmission to the control means, of any measurement of respiratory gas flow by the flow sensor (i.e. in the case of loss of the respiratory flow signal), the control means are configured to control the control means of the main and/or secondary flow(s) in order to supply the gas containing NO to an emergency flow obtained or calculated from one or a plurality of set gas flows containing NO, which have been determined by the control means and stored by the storage means, before the said interruption of transmission.

According to the embodiment concerned, the installation according to the invention can comprise one or a plurality of the following characteristics:
the control means are configured to determine successive set gas flows containing NO from a plurality of successive respiratory gas flow measurements which are carried out by the flow sensor and supplied to the said control means.
the storage means are configured to store successive set gas flows containing NO which have been determined by the control means.
the storage means are configured to store successive the set gas flows containing NO which have been determined during the normal operation of the apparatus and/or the installation, i.e. before any loss of the respiratory flow signal.
the emergency flow is obtained or calculated from a plurality of set flows which have been determined by the control means during a given period (dt), before the said interruption of transmission, i.e. successive flows determined during the normal operation of the apparatus.
the emergency flow is calculated by obtaining a mean of the set flows which have been stored throughout the given period (dt), i.e. before any loss of signal of the respiratory flow.
the given period (dt) is 30 seconds or less, preferably 20 seconds or less, or more preferably 10 seconds or less.
the given period (dt) is fixed, or, according to another embodiment, modifiable.
the given period (dt) is stored.
the control means are configured to determine the said at least one set gas flow containing NO to be supplied to the injection device, from at least one respiratory gas flow measurement carried out and supplied by the flow sensor, of a set NO content corresponding to the final proportion of NO required (i.e. a posology) in the combined gaseous mixture, and from the initial proportion of NO in the gas containing NO which supplies the NO supply apparatus.
the initial proportion of NO in the gas containing NO which supplies the NO supply apparatus is stored within storage means.
in the case of interruption of transmission to the control means, of any measurement of respiratory gas flow by the flow sensor (i.e. in the case of loss of the respiratory flow signal), the control means are configured to control the main flow control means (i.e. which remain operational, in other words the main flow control means which are not malfunctioning), in order to supply the gas containing NO to the said emergency flow.
when the main flow control means continue to operation normally (i.e. which are not malfunctioning), despite the interruption of transmission of the flow measurements (i.e. in the case of loss of the respiratory flow signal), the gas containing the NO passes via the main line comprising the main flow control means which are controlled by the control means, in order to supply the gas containing NO to the said emergency flow.
on the contrary, in the case of interruption of transmission to the control means of any respiratory gas flow measurement by the flow sensor, and also malfunctioning of the main gas control means (therefore of the main flow control means which are non-operational or out of service), the control means are configured to control the secondary flow control means (in order to make them operational), so as to supply the gas containing NO to the said emergency flow.
when the main flow control means are not operating normally, i.e. when they are malfunctioning, moreover with interruption of transmission of the flow measurements (i.e. in the case of loss of the respiratory flow signal), the gas containing the NO passes via the secondary line (and no longer via the main line), comprising the secondary flow control means which are controlled by the control means in order to supply the gas containing NO to the said emergency flow.
the flow sensor is arranged within the respiratory circuit, upstream from the injection device.
the NO supply apparatus is supplied with a gas containing an initial proportion of NO of between 100 and 1500 ppmv, typically between 200 and 1000 ppmv.
the NO supply apparatus is supplied with a gaseous mixture formed by nitrogen and NO.
the NO supply apparatus comprises dose regulation means which are configured to allow a user to set or select the set NO content corresponding to the final proportion of NO required in the combined gaseous mixture, i.e. a posology.
the dose regulation means form part of an HMI (man-machine interface) or UGI (user graphics interface).
the dose regulation means comprise one or more touch keys which can be actuated by the user, displayed on a digital touch screen of the UGI.
the HMI screen is of the colour display type.
the set NO content is between 1 and 80 ppmv, typically between 5 and 40 ppmv.
it comprises a medical ventilator which is configured to supply the flow of respiratory gas containing O₂ to the said respiratory circuit.
the medical ventilator is configured to supply a flow of respiratory gas containing at least 20% by volume approximately of O₂, typically a mixture of NO/N₂ or air.
the NO supply apparatus and the medical ventilator are connected fluidically to the respiratory circuit.
the flow sensor is arranged in the respiratory circuit between the medical ventilator and the injection device.
the injection device also comprises a first gas input which is supplied with respiratory gas flow containing O₂, i.e. coming from the medical ventilator.
the injection device also comprises a second gas input which is supplied with gas containing NO at the said set flow, i.e. coming from the NO supply apparatus.
the injection device also comprises a gas output supplying the combined gaseous mixture containing NO and oxygen, obtained by mixing, within the injection device, of the gas containing NO (e.g. NO/N₂ mixture) with the flow of respiratory gas containing O₂ (e.g. air or O₂/N₂ mixture).
the storage means comprise a computer memory, such as flash memory, a RAM or the like. P1 the control means are configured to detect any interruption of transmission of the respiratory gas flow measurements by the flow sensor to the control means.
the control means are configured to detect any interruption of transmission of the respiratory gas flow measurements made by the flow sensor, i.e. any loss of the respiratory flow signal.
the NO supply apparatus operates according to at least two operating modes comprising a normal operating mode and an emergency operating mode, i.e. an emergency mode.
the NO supply apparatus is configured to go automatically from the normal operating mode to the emergency mode in response to an interruption of transmission of the respiratory gas flow measurements carried out by the flow sensor (i.e. in the case of loss of the respiratory flow signal), with or without malfunctioning of the main flow control means, in particular of the main valve means.
detection by the control means is configured to detect any interruption of transmission of the respiratory gas flow measurements and/or any malfunctioning of the main flow control means.
the control means are configured to activate the emergency mode in the case of interruption of transmission of the respiratory gas flow measurements (i.e. in the case of loss of the respiratory flow signal).
the control means are configured to determine the successive set gas flows containing NO from successive respiratory gas flow measurements carried out by the flow sensor during normal operation of the NO supply apparatus, i.e. when it is operating according to the normal operating mode.
the control means are configured to control the main and/or secondary flow control means in order to supply the gas containing NO to the emergency flow, during operation of the NO supply apparatus in emergency mode.
the control means are configured to determine and store the successive set flows containing NO during the normal operation of the NO supply apparatus.
the internal gas circuit comprises two gas sections which are arranged in parallel, comprising a main section and a secondary section or emergency section.
the main and secondary sections comprise main and secondary flow control means, typically main and secondary valve means, such as solenoid valves or the like.
the control means are configured to control the main and secondary flow control means in order to permit or prevent the passage of the flow of NO/N₂ in one or the other of the main and secondary sections, in particular according to the operating mode of the NO supply apparatus.
in the normal operating mode, the control means are configured to control the main and/or secondary flow control means in order for the flow of NO/N₂ to pass only via the main section.
in emergency mode, the control means are configured to control the main and/or secondary flow control means so that the flow of NO/N₂ passes via the main section, or, depending on the case, via the emergency section, according to whether the main flow control means are operating, or on the other hand are malfunctioning, respectively.
the main and secondary flow control means comprise main and secondary valve means, such as solenoid valves.
the main flow control means comprise a proportional solenoid valve.
the secondary flow control means comprise an all-or-nothing solenoid valve, which is preferably controlled in pulse mode.
the main flow control means comprise a mass flow controller.
the mass flow controller or MFC comprises at least one proportional solenoid valve which is controlled by the control means, and a main flow sensor.
the main flow sensor is incorporated in the structure of the proportional solenoid valve of the MFC.
the MFC solenoid valve also comprises an electronic microprocessor board which implements at least one algorithm.
the secondary section, i.e. the emergency section, comprises a calibrated orifice device which is used to control the flow of gas circulating in the secondary section, in particular in emergency mode.
the calibrated orifice device is arranged downstream from secondary flow control means of the secondary section.
the secondary flow control means of the secondary section comprise secondary valve means, typically a solenoid valve, preferably of the all-or-nothing type (AON).
the solenoid valve of the all-or-nothing type (AON) is controlled by the control means, preferably in pulse mode.
the main section comprises one (or more) proportional solenoid valve(s), and the secondary section comprises one (or more) solenoid valve(s) of the all-or-nothing (AON) type.
the main section comprises one (or more) proportional solenoid valve(s) which is/are normally closed.
in normal operation, the proportional solenoid valve is controlled so as to be at least partly open (i.e. proportional opening), and allow gas containing NO to pass.
   in normal operation, the AON solenoid valve is closed or controlled by the control means so as to be closed, and prevent any passage of gas containing NO, i.e. to prevent the circulation of gas.
in the case of malfunctioning of the main flow control means, typically of the MFC, the proportional solenoid valve closes automatically, or, depending on the case, is controlled such as to close, so as to prohibit, stop or prevent any circulation of gas.
in the case of malfunctioning of the main flow control means, typically of the MFC, the AON solenoid valve opens automatically, or is controlled so as to be opened (i.e. to open), such as to authorise or permit a passage of gas (e.g. NO/N₂) in the secondary section.
the control means comprise a (micro)controller or the like.
the control means comprise one (or more) (micro)processor(s) arranged on one (or more) electronic board(s).
the control means comprise one (or more) (micro)processor(s) which implement one or more algorithm(s), in particular one (or more) algorithm(s) for controlling main and/or secondary flow control means, one (or more) algorithm(s) for processing of respiratory gas measurements etc.
the storage means are incorporated in the control means, and in particular are arranged on the electronic board.

In addition, according to the embodiment concerned, the installation for supply of gas according to the invention can comprise one or a plurality of the following additional characteristics:
the medical ventilator supplies air or an oxygen/nitrogen mixture, i.e. as a respiratory gas containing at least 20% by volume approximately of oxygen, preferably at least 21% by volume approximately of oxygen.
the medical ventilator comprises a motorised blower (i.e. turbine, compressor or the like) which supplies the respiratory gas, typically air or an oxygen/nitrogen mixture or, according to another embodiment, an internal gas circuit comprising one or more proportional valves to convey the gas and control its supply, in particular its flow. Such a ventilator is generally supplied with respiratory gas via one (or more) wall outlet(s) supplied with gas from a network of ducts in a hospital establishment or building, typically air or an oxygen/nitrogen mixture.
the medical ventilator comprises control means or a device, such as one (or more) electronic control board(s). Preferably the control means of the medical ventilator control or command the motorised blower, or, depending on the case, the proportional valves of the medical ventilator.
the medical ventilator is of the HFO type or comprises an HFO function, that is to say it is able to produce high-frequency oscillations.
the source of NO contains an NO/N₂ gaseous mixture containing between 100 and 2,000 ppmv of NO, the remainder being nitrogen (N₂), preferably between 100 and 1000 ppmv of NO, conditioned at a pressure of between 10 and 250 bars abs, typically at more than 100 bars abs (before the start of drawing off).
the source of NO is or comprises one (or more) gas cylinder(s) with a content of between 0.5 and 50 L (equivalent in water).
the gas cylinder(s) comprise(s) a cylindrical body made of steel or aluminium alloy, and is equipped with a simple tap (without a pressure-reducing valve) or with an incorporated pressure-reducing valve or RDI, preferably an RDI, protected by a protective cover, which for example is made of metal or polymer.
the respiratory circuit of the installation comprises an inhalation branch and an exhalation branch, typically flexible ducts forming the inhalation branch and the exhalation branch, for example polymer pipes.
the inhalation branch and the exhalation branch, e.g. flexible ducts, are connected to a joining part, such as a part in the form of a “Y”.
the inhalation branch and/or the exhalation branch are connected fluidically to a patient respiratory interface, preferably via the joining part.
the patient respiratory interface comprises a tracheal intubation sensor or a respiratory mask, or the like.
the inhalation branch and the exhalation branch are moreover fluidically connected to output and input orifices, respectively, of the medical ventilator.
the respiratory circuit, in particular the inhalation branch, can comprise a gas humidifier.
the gas humidifier is arranged downstream from the injection device, for example an NO injection module, such as to be able to humidify the gas before it is administered to the patient by inhalation.
the ventilator and the NO supply apparatus are supplied electrically by one or more electric current source(s), typically the mains supply (110/220 V) and/or one or more rechargeable battery/batteries.

According to another aspect, the invention also concerns a method for therapeutic treatment of a person, i.e. a human patient (i.e. an adult, child, adolescent or newborn), suffering from pulmonary hypertension and/or hypoxia, giving rise to pulmonary vasoconstrictions or the like, comprising administration by inhalation to the person needing it, of a gaseous mixture comprising from 1 to 80 ppmv of NO and at least 20% by volume of oxygen approximately, preferably at least 21% by volume of oxygen approximately, by means of a gas supply installation, such as the one previously described according to the invention, comprising an NO supply apparatus which assures a supply of NO to an emergency flow compatible with the required posology, even in the case of malfunctioning which prevents any exchange of measurements between the flow sensor measuring the flow of respiratory gas based on oxygen supplied by the medical ventilator (e.g. air or N₂/O₂) and the means for controlling the NO supply apparatus, such as to treat (at least partly) the said pulmonary hypertension and/or the said hypoxia, which can be caused by one (or more) pathology/pathologies or other pulmonary troubles, typically of the PPHN (persistent pulmonary hypertension of the newborn) or ARDS (acute respiratory distress syndrome) type, or also generated by a cardiac surgery operation when the patient is subjected to extra corporeal blood circulation (EBC).

### DEFINITIONS

In general, within the context of the invention:
“ppmv” means parts per million by volume.
“% vol.” means percentage by volume.
“NO” designates nitrogen monoxide.
“NO₂” designates nitrogen dioxide.
“N₂” designates nitrogen.
“O₂” designates oxygen.
the terms “concentration”, “quantity”, “proportion”, “dose” and “content” are considered as equivalents.
the terms “means of/at/for” are considered as totally equivalent and can be substituted by the terms “device of/at/for”, for example the term “control means” can be replaced by “control device”, the term “valve means” can be replaced by “valve device”, “storage means” can be replaced by “storage device”, etc.
“normal operation” or “normal operating mode” mean habitual operation of the NO supply apparatus in the absence of any malfunctioning impeding or preventing a supply (i.e. interruption of transmission or loss of flow signal) to the means for controlling the NO supply apparatus, of the flow measurements (i.e. values or signals) by the flow sensor measuring the flow of the respiratory gas (i.e. air or N₂/O₂) conveyed by the respiratory circuit, typically coming from the medical ventilator.
“malfunctioning” means a fault, an abnormality, a problem, a defect, an accidental disconnection or any other technical problem, such as, for example, electromagnetic disturbances, which affect the normal operation of the NO supply apparatus by preventing the supply to the control means and/or the receipt by the said control means (i.e. interruption of transmission of the signal or loss of the flow signal), of the flow measurements carried out by the flow sensor measuring the flow of the respiratory gas (i.e. air or N₂/O₂) conveyed by the respiratory circuit, typically coming from the medical ventilator.
“flow measurement” means a flow value (e.g. a numerical value) or a signal representative of such a flow value, which reflects or corresponds to a gaseous flow measured by a flow sensor, such as a mass flow sensor.
“pressure measurement” means a pressure value (e.g. a numerical value) or a signal representative of such a pressure value, which reflects or corresponds to the gaseous pressure measured by a pressure sensor (irrespective of the operating mode of this sensor).
“operation in emergency mode” means operation of the NO supply apparatus in the case of malfunctioning impeding or preventing the supply (i.e. interruption of transmission of the flow signal or loss of the flow signal) to the means for controlling the NO supply apparatus, of the flow measurements of the respiratory gas flow (i.e. air or O₂/N₂ mixture) carried out by the flow sensor, with or without any concomitant malfunctioning of the main flow control means (i.e. of the proportional solenoid valve of the MFC) situated on the main gas section.

### BRIEF DESCRIPTION OF VIEWS OF DRAWINGS

The invention will now be better understood thanks to the following detailed description, provided by way of non-limiting illustration, with reference to the appended figures, in which:

FIG. 1 schematises an embodiment of a gas administration installation according to the invention.

FIG. 2 schematises an embodiment of the internal architecture of the NO supply apparatus of a gas administration installation according to the invention, in particular a gas administration installation according to FIG. 1.

### DETAILED DESCRIPTION

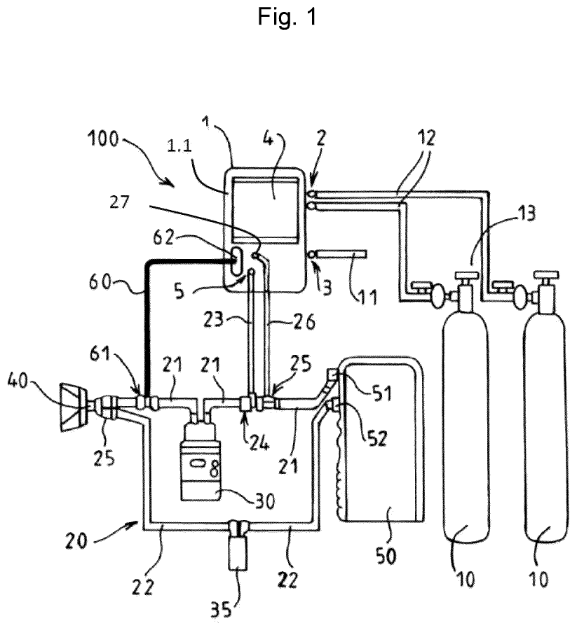

FIG. 1 schematises an embodiment of a gas administration installation 100 according to the invention, comprising an NO supply apparatus 1 which supplies a gaseous mixture based on nitrogen monoxide (NO), and a medical ventilator 50 which supplies gas containing at least 20% by volume of oxygen, such as air or the like.

In this case, the installation 100 comprises two pressurised gas cylinders 10, each containing a NO/N₂ gaseous mixture, in this case an NO/N₂ gaseous mixture containing between 100 and 1000 ppmv of NO (remainder N₂), for example 450 or 800 ppmv of NO (remainder N₂), or any other adequate concentration which supplies with NO/N₂ mixture the device or apparatus 1 for delivery or supply of NO making it possible to monitor and control the supply of the NO/N₂ gaseous mixture.

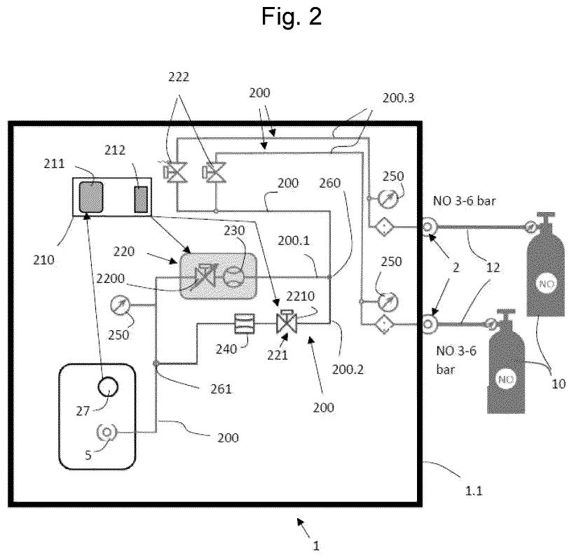

The gas cylinders 10 are fluidically connected to the NO supply apparatus 1 via gas feed lines 12, such as flexible pipes or ducts or the like, which may be equipped with devices for regulating and/or monitoring the gas pressure, such as a gas pressure-relieving valve 13, pressure gauges, etc. The gas feed lines 12 are connected to one or a plurality of gas inputs 2 of the NO supply apparatus 1, which supply an internal gas circuit 200, as schematised in FIG. 2, used to convey the gas within the NO supply apparatus 1, i.e. into the external casing 1.1 of the apparatus 1.

In the embodiment of FIG. 2, the internal gas circuit 200 is connected to two gas inputs 2 which are arranged in parallel, and each supply an input section 200.3 dedicated to the internal gas circuit 200. Control valves 222 or the like control the passage of the flow of NO/N₂ in these input sections 200.3.

The NO supply apparatus 1 also comprises an oxygen input 3, which is connected fluidically, via an oxygen feed line 11, such as a flexible pipe or the like, to a source of oxygen (not shown), for example a pressurised oxygen cylinder or a hospital network, i.e. an oxygen supply duct which is provided in a hospital building. This makes it possible to supply the internal gas circuit 200 with oxygen when necessary.

The medical ventilator 50, i.e. a respiratory assistance apparatus, supplies a flow of respiratory gas based on oxygen, i.e. containing at least 20% by volume of oxygen approximately, preferably at least 21% by volume of oxygen approximately, such as air or a mixture of oxygen/nitrogen (N₂/O₂).

The medical ventilator 50 and the NO supply apparatus 1 of the installation 100 are in fluidic communication with a respiratory circuit 20, also known as the patient circuit, in particular with a gas supply line or inhalation branch 21 of the respiratory circuit 20, which is used to convey the gaseous flow to the respiratory interface 40 which supplies the therapeutic gaseous flow to the patient, i.e. a final gaseous mixture containing the required posology of NO.

More specifically, the final gaseous mixture to be administered to the patient is formed by mixing the flow based on oxygen (i.e. air or NO/N₂ mixture) coming from the medical ventilator 50, and the flow containing the NO, i.e. the NO/N₂ gaseous mixture supplied by the NO supply apparatus 1.

For this purpose, the NO supply apparatus 1 supplies or injects the NO/N₂ mixture into the respiratory circuit 20, typically into the respiratory branch 21, via a channel or an injection line 23, which connects fluidically the internal gas circuit of the NO supply apparatus 1 to an injection device 24 arranged on the gas supply line 21.

The injection device 24 is configured to mix gas containing NO coming from the NO supply apparatus 1 with the flow of respiratory gas containing O₂ coming from the ventilator 50, and conveyed by the inhalation branch 21 of the respiratory circuit 20, and obtain a combined gaseous mixture containing NO and oxygen, i.e. the final gaseous mixture administered to the patient.

More specifically, the injection device 24 comprises a first gas input supplied with a respiratory gas flow containing O₂ coming from the medical ventilator 50, a second gas input supplied with gas containing NO, i.e. coming from the NO supply apparatus 1, and a gas output supplying the combined gaseous mixture containing NO and oxygen, obtained by mixing, within the injection device 24, of the gas containing NO with the flow of respiratory gas containing O₂.

In other words, the flow of NO/N₂ fed by the injection line 23 is then mixed (thanks to the injection device 24) with the flow of gas based on oxygen (>20% O₂), e.g. air or oxygen/nitrogen mixture, supplied by the medical ventilator 50 and conveyed by the inhalation branch 21 of the patient circuit 20, so as to obtain a final mixture, i.e. a combined mixture, to be administered to the patient, containing substantially NO at the required pathology, nitrogen (N₂) and oxygen (O₂), and possibly inevitable impurities (e.g. argon, CO₂, NO₂, etc.), i.e. a final NO/N₂/O₂ gaseous mixture.

The inhalation branch 21 of the circuit 20 also comprises a gas humidifier 30 arranged downstream from the injection device 24. It makes it possible to humidify the final flow of gas, i.e. the combined NO/N₂/O₂ gaseous mixture before it is administered by inhalation to the patient to be treated, by means of a respiratory interface 40, such as a tracheal intubation sensor, a respiratory mask or the like.

A line for recuperation of the gases exhaled by the patient forms an exhalation branch 22 of the patient circuit 20. It is connected fluidically to the inhalation branch 21 via a connection part 25, such as a part in the form of a “Y”.

At its upstream end, the inhalation branch 21 is connected fluidically to an output port 51 of the medical ventilator 50, such as a connector, connection or the like, such as to recuperate and convey the gas based on oxygen, typically air or N₂/O₂ mixture supplied by the medical ventilator 50, whereas the exhalation branch 22, which conveys the exhaled gases, is connected fluidically to an input port 52 of the medical ventilator 50, such as a connector, connection or the like, such as to return to the medical ventilator 50 all or part of the flow of the gases exhaled by the patient. The exhalation branch 22 can comprise one or a plurality of optional components, for example a CO₂ removal device 35, i.e. a CO₂ trap, such as a hot container or the like, making it possible to remove the CO₂ present in the gases exhaled by the patient, or a filter or the like.

In addition, a flow sensor 25, for example of the hot wire or pressure-differential type, is arranged on the respiratory circuit 20, in particular on the inhalation branch 21, between the ventilator 50 and the injection device 24. The flow sensor 25 is connected at a connection port to the sensor 27 of the NO supply apparatus 1 via one (or more) flow measurement line(s) 26 which is/are connected at the said connection port to the sensor 27. It is used to measure the flow of gas supplied by the ventilator 50, such as air or N₂/O₂, circulating in the inhalation branch 21, upstream from the injection device.

These flow measurements which are carried out by the flow sensor 25 make it possible to control or regulate more efficiently the supply of the flow of NO (i.e. N₂/O₂) by the NO supply apparatus 1, in particular the flow of NO, since the flow measurements carried out by the flow sensor 25 are returned, via the flow measurement line 26 (i.e. electric cables or the like) and the port for connection to the sensor 27, to (micro)processor control means 210 of the NO supply apparatus 1, typically a controller, which process these flow measurements as explained hereinafter and illustrated in FIG. 2. The port for connection to the sensor 27 is connected electrically to the control means 210 via one or more electrical connections, for example electrical cables or the like.

The NO supply apparatus 1 comprises a rigid casing 1.1, which for example is made of polymer, comprising the internal gas circuit 200 in FIG. 2, typically gas lines, passages or ducts or the like, used to convey the flow of gas based on NO, i.e. the NO/N₂ mixture, coming from the NO/N₂ mixing cylinders 12. The internal gas circuit 200 connects the gas input (or inputs) 21 of the NO supply apparatus 1 fluidically to the injection line 23, such as to convey the flow of gas based on NO between them.

In the embodiment schematised in FIG. 2, a portion of the internal gas circuit 200 comprises two gas sections arranged in parallel, i.e. a main section 200.1 and a secondary section 200.2, known as the emergency section The main section 200.1 and the secondary section 200.2 are connected fluidically to one another and to the remainder of the gas circuit 200, at upstream 260 and downstream 261 connection sites, which are situated respectively upstream and downstream from the main and secondary flow control means 220, 221.

In this case, in normal operating mode, the flow of NO/N₂ passes via the main section 200.1, whereas in the case of interruption of transmission of the signal, it goes automatically into emergency mode, as explained hereinafter and:
if the main flow control means 220 remain operational, i.e. if they continue to operate normally, typically a mass flow controller or MFC, then the flow of NO/N₂ continues to pass via the main section 200.1;
if the main flow control means 220 are rendered non-operational, i.e. dysfunctional, the flow of NO/N₂ is diverted, and then passes via the emergency section 200.2.

It will be appreciated that, according to another embodiment (not shown), the internal gas circuit 200 could be configured differently, for example it could comprise a single gas line instead and in the place of the two sections 200.1, 200.2, which line would be used in normal operating mode and in emergency mode. However, in this embodiment, malfunctioning of the main flow control means 220 could not be taken into consideration, and the apparatus 1 would then become non-functional.

In general, the main and secondary flow control means 220, 221, such as main and secondary valve means 2200, 2210, schematised in FIG. 2, i.e. one (or more) valve device(s), for example one (or more) proportional solenoid valve(s) controlled by the control means 210, are arranged on the internal gas circuit 200, in particular on the main 200.1 and secondary 200.2 sections, and are used to control or adjust the gaseous flow circulating therein in the direction of the injection line 23, i.e. towards the injection device 24, irrespective of whether this is in normal operating mode or in emergency mode.

Preferably, the main section 200.1 comprises a proportional solenoid valve 220 and an additional flow sensor 230, typically a mass flow controller or MFC, whereas the secondary section 200.2 comprises one (or more) solenoid valve(s) of the all-or-nothing (AON) type 221, preferably controlled in pulse mode.

Preferably, the main and secondary flow control means 220, 221 of the NO supply apparatus 1 are commanded, i.e. controlled, by the control means 210, i.e. one (or more) control device(s) or (micro)controller(s), arranged in the housing 1.1 of the NO supply apparatus 1.

Typically, the control means 210 comprise one (or more) electronic board(s) comprising one (or a plurality of) microprocessor(s) 211 implementing one or more algorithms. The control means 210 make it possible in particular to adjust or control the flow of gas based on NO by controlling all or part of the valve means 2200, 2210, typically to open or close one or more (solenoid) valve(s), in order to obtain a gas flow based on NO, typically to permit or stop the flow of gas.

It will be appreciated that the control means 210 also make it possible to carry out calculations, and/or to control or command all the electromechanical elements of the apparatus 1, such as sensors, displays, etc.

As explained hereinafter, the control means 210 can determine the flow of NO to be supplied in order to obtain the NO content required in the combined mixture, i.e. the desired posology of NO, in particular on the basis of the set NO content regulated and/or set by the user, of the composition of the gaseous NO/N₂ mixture, in particular of the NO content in this NO/N₂ gaseous mixture, and one (or more) flow measurement(s) carried out by the flow sensor 25 arranged on the inhalation branch 21, and connected by a flow measurement line 26 to the NO supply device 1, in particular to the control means 210, via the port for connection to the sensor 27.

The internal gas circuit 200 of the NO supply apparatus 1 can also comprise other elements or components, in particular one (or more) pressure sensor(s) 250, one (or more) additional flow sensor(s) or flow meter(s), and/or calibrated orifice devices 240 or the like. These other elements can be arranged upstream and/or downstream from the flow control means 220, 221, i.e. valve means, for example it is possible to use an additional flow sensor in order to determine the flow of gas based on NO circulating in all or part of the internal gas circuit 200, in particular in order to assure that it is in conformity with the required flow.

Thus, in FIG. 2, it can be seen that the main section 200.1 comprises an additional flow sensor 230, arranged upstream from the flow control means 220, such as valve means 2200, for example one (or more) solenoid valve(s), preferably a proportion solenoid valve, controlling the passage of gas in the main section 200.1. This assembly forms a mass flow controller (MFC).

In addition, the secondary section 200.2 comprises a calibrated orifice device 240 arranged downstream from secondary flow control means 221, such as secondary valve means 2210, preferably one (or more) solenoid valve(s) controlling the flow of passage of gas in the secondary section 200.2.

Advantageously, the solenoid valve of the secondary flow control means 221 is of the all-or-nothing (AON) type, i.e. which can adopt 2 “stable” positions, in other words an open position which allows the gaseous flow to pass, and a closed position which prevents any circulation of gaseous flow.

According to the invention, during an interruption of transmission of the respiratory gas flow measurement signal, i.e. a loss of the respiratory gas flow signal, but with main flow control means 220 (MFC) which are operational, i.e. which continue to operate normally, the flow of NO/N₂ continues to travel via the main section 200.1, and the control means 210 command the proportional solenoid valve 220, i.e. in proportional mode, to adjust and supply the gas to the required emergency flow. The solenoid valve of the all-or-nothing (AON) type 221 provided on the secondary section 200.2 is for its part then in the closed position The apparatus 1 is thus in emergency mode, but the flow of NO/N₂ continues to travel via the main section 200.1.

On the other hand, in the case of interruption of transmission of the respiratory gas flow measurement signal with simultaneous malfunctioning of the main flow control means 220 (i.e. of the MFC, for example of the proportional solenoid valve), the control means 210 then command (typically in pulse mode) opening of the solenoid valve of the all-or-nothing (AON) type 221, provided on the secondary section 200.2, in order to allow the gas to pass into this secondary section 200.2, so as to supply the gas to the required emergency flow, whereas the proportional solenoid valve 220 is no longer controlled, and goes into the closure position, which is preferably its default position. The apparatus 1 is then also in emergency mode, and the flow of NO/N₂ no longer passes via the main section 200.1.

In addition, the flow meter or additional flow sensor 230 of the MFC can be of the pressure differential, mass, or another type, and cooperates with the control means 210 in order to provide them with flow measurements of the flow of NO/N₂.

Habitually, the NO supply apparatus 1 also comprises a graphic user interface (GUI) comprising a graphic display screen 4, preferably a touch screen, i.e. with a touch panel, used to display various information items or data, icons, curves, alerts, etc., and also virtual selection keys and/or panes or windows, in particular for making choices, selections or for entering information, such as desired values (e.g. flow, dose of NO, etc.), or any other information or data useful to the medical personnel. The display is preferably colour, but it can also be in black and white.

The electrical supply of the NO supply apparatus 1, in particular of the components which require electric current in order to operate, such as the control means 210, the graphic display 4, etc., is provided conventionally by a source of electric current and/or electrical supply means (not shown), for example by means of a connection to the mains current (110/220 V) of the electric cord and connection socket type, and/or one (or more) electrical supply battery/batteries, which is/are preferably rechargeable, and/or a current transformer. The electrical power supply to the medical ventilator 50 is assured in a similar manner, in particular by a connection to the mains current or by an internal battery.

In addition, the installation 100 also comprises a gas collection line 60, which connects the inhalation branch 21 fluidically to the NO supply apparatus 1. It is connected fluidically (at 61) to the gas supply line 21, between the humidifier 30 and the joining part 25, i.e. the part in the form of a “Y”, typically in the immediate vicinity of the joining part 25, and also to an input port 62 of the NO supply device 1, for example a port 62 which is carried by a connector, connection or the like, permitting the connection of the gas collection line 60, such as a flexible pipe or the like. The gas collection line 60 makes it possible to collect samples of gas and convey them to the NO supply line 1, where they are analysed in an internal gas analyser (not shown), i.e. within a calibration line comprising at least one sensor, in particular one or more electrochemical cell(s) connected electrically to the control means, in order to verify the conformity thereof. In particular, it must be verified that the composition of the final gas is in conformity with that of the required NO/N₂/O₂ gaseous mixture to be administered to the patient, in particular in order to assure that it does not contain an excessive quantity of toxic NO₂ substances, that its oxygen content is not hypoxic, that it does not contain an excessively high content of NO₂, and that its NO content corresponds to the required posology, i.e. the dose of NO to be administered by inhalation which is habitually selected by the medical staff, i.e. the doctor or the like. This verification of conformity is conventionally carried out by means of dedicated measurement means, typically sensors for NO₂, NO and O₂, for example electrochemical cells or the like, which themselves must be calibrated periodically, for example every week. The control means 210 of the apparatus 1 are also configured to recuperate and process, i.e. analyse, the signals coming from the different gas analyser sensors, which is provided in the apparatus 1, and to act in response to these signals, in particular in order to carry out calibration of the sensors.

Within the context of the invention, in order to be able to determine a set NO flow, in the absence of measurement of a respiratory gas flow, i.e. in the case of interruption of the transmission to the control means 210 or loss of signal, of the respiratory gas flow measurements carried out by the flow sensor 25, and consequently in order to be able to supply a final gaseous mixture based on NO, i.e. a combined mixture, to the patient, containing a proportion of NO equal or close to a posology set by the medical staff, i.e. a doctor or the like, the following procedure is carried out.

The interruption of transmission of the flow measurements, i.e. the loss of signal, can be derived from malfunctioning or a defect of the flow sensor, loss of electrical connectivity, such as accidental disconnection of the flow sensor, or sectioning of a connection cable, or from another cause, such as electromagnetic disturbances or the like.

During the normal operation of the apparatus 1, i.e. before any interruption of transmission of flow measurement coming from the flow sensor 25, the (micro)processor 211 control means 210, such as a controller, determine the set gas flow containing NO to be supplied to the injection device 24, and flow control means 220, 221 such as proportional 220 and/or AON 221 solenoid valves, in order to supply gas containing NO to the set flow which has been determined, as has previously been explained.

This determination of the set NO flow is carried out from a respiratory gas flow measurement, i.e. a flow signal or value, carried out and supplied by the flow sensor 25 to the control means 210, of a set NO content corresponding to the final proportion of NO required in the combined gaseous mixture, typically set by the user, i.e. the medical staff, and of the initial proportion of NO in the gas containing NO supplying the NO supply apparatus 1, i.e. the quantity of NO present in the NO/N₂ mixture coming from the gas cylinders 10, typically of between 200 and 1000 ppmv, for example 450 or 800 ppmv.

The set NO value and/or the NO content in the NO/N₂ gaseous mixture supplying the apparatus 1 can be entered and/or adjusted and/or modified by the user, for example via the HMI, thanks to means for regulation of the dose or the like, such as keys, cursors, and so on. Preferably, the set NO value and/or the content of NO in the NO/N₂ gaseous mixture supplying the apparatus 1 can be stored by the storage means 212 of the apparatus 1.

Advantageously, the set NO flow is determined, i.e. updated, at a frequency of between 10 Hz and 1000 Hz, or in a temporal period of between 1 and 100 ms (msec).

The successive set NO flows thus determined during the normal operation of the apparatus 1 are stored by storage means 212, i.e. a storage device, such as a flash memory or the like. These set NO flows are used to control the flow control means 220, 221 provided on the internal circuit 200, in particular on the main section 200.1 and the secondary section 200.2, i.e. the emergency section, as explained above, in order to supply the gas containing NO to the set flow which has been determined.

As previously stated, in normal operating mode, in the embodiment of FIG. 2, the flow of NO/N₂ passes via the main section 200.1, since the main flow control means 220, typically main valve means 2200, of the main section 200.1, are in the open position, in order to allow the gaseous flow to pass, whereas the secondary flow control means 221, i.e. the secondary valve means 2200, of the secondary section 200.2, are in the closed position, in order to prevent any circulation of gas in the secondary section 200.2, i.e. to block or prevent any circulation of gaseous flow based on NO within this secondary section 200.2.

According to the invention, in the case of interruption of transmission to the control means 210, of any respiratory gas flow measurement by the flow sensor 25, the NO supply apparatus 1 goes into emergency mode, and the control means 210 are then configured to control, command or order the main and secondary flow control means 220, 221, typically the main and secondary valve means 2210, 2200, to supply the gas containing NO to an emergency flow obtained or calculated from one or more set gas flow(s) containing NO determined by the control means 210, and stored by the storage means 212, before the said interruption of transmission.

In the embodiment of FIG. 2, in the case of interruption of transmission to the control means 210 of any respiratory gas flow measurement by the flow sensor 25, the flow of NO/N₂ then passes via the main 200.1 or via the secondary 200.2 sections, depending on whether the main flow control means 220, i.e. the main valve means 2200 of the main section 200.1 are also malfunctioning or not, as previously explained.

Thus, when the main flow control means 220 of the main section 200.1, such as a proportional solenoid valve of an MFC, are operating normally, the control means 210 are configured to control the main and/or secondary flow control means 220, 221, in order to make the gaseous flow circulate through the said main flow control means 220, and supply the flow of emergency gas via the main section 200.1.

On the other hand, in the case of malfunctioning also of the main flow control means 220 of the main section 200.1, such as a proportional solenoid valve, the said main flow control means 220, i.e. the main valve means 2200, of the main section 200.1, go (by default) into the closed position in order to stop the gaseous flow, whereas the secondary flow control means 221, i.e. the secondary valve means 2100, of the secondary section 200.2, such as an AON solenoid valve, are commanded by the control means 210, to be in the open position, and thus permit circulation of gas in the secondary section 200.2, and supply thereof to the emergency gas flow, after passage into the calibrated orifice device 240.

In other words, the NO supply apparatus 1 is thus designed to operate according to (at least) two operating modes, i.e. a so-called “normal” operating mode and a so-called “emergency” operating mode, known as the “emergency mode”. The passage from the normal operating mode to the emergency operating mode takes place automatically in response to detection by the control means 210 of an interruption of transmission of the respiratory gas flow measurements carried out by the flow sensor 25.

In all cases, in the case of interruption of transmission of the signal, the emergency flow is obtained or calculated from a plurality of set flows which have been previously determined and stored, during a given period dt, by the control means 210, during the normal operation of the apparatus 1, i.e. before the said interruption of transmission and passage into emergency mode.

The period dt corresponds to the period of time preceding the moment of the interruption of transmission of the flow signal, i.e. the detection by the control means 210 of the interruption of transmission of the flow measurements. Preferably, the period dt is 30 seconds or less, preferably 20 seconds or less, for example 10 seconds or less.

In other words, use is not made of all the set flow values which have been determined and stored during the normal operation of the apparatus 1, which generally lasts for several hours, but only the most recent ones, i.e. those determined and stored during the period dt, i.e. the short period of time, typically <30 seconds, which immediately precedes the moment or instant where the interruption of transmission of the measurements (i.e. signal or value) of flow has taken place and has been detected by the control means.

The set flow values which have been determined and stored during this period dt are preferably averaged, and optionally weighted or the like, in order to obtain a mean flow value, i.e. the emergency flow.

It is thus understood that, according to the invention, the emergency flow value(s) is/are calculated from set NO flow values which have previously been used to control the flow of NO, i.e. the flow, during the normal operation of the apparatus 1, and stored by the storage means of the apparatus 1, during a period dt, contrary to what is taught by the prior art, which, in the case of interruption of transmission of the flow signal, advocates either a single prefixed constant emergency flow value, or flow values of the respiratory gas flow (e.g. air or N₂/O₂), or the flow of NO measured during the normal operation of the apparatus 1, and recorded in a stored history.

In other words, before the present invention, it has never been advocated to store and use set NO flow values which have previously been used to control the flow of NO, during the normal operation of the apparatus 1, in order to use them subsequently in the case of interruption of the transmission of the flow measurements (i.e. loss of the flow signal) coming from the respiratory flow sensor, e.g. air or O₂/N₂, i.e. after passage into emergency mode.

According to the invention, the emergency flow preferably corresponds to a mean flow value obtained from set flow values determined and stored, during the period dt, in general a period dt of less than 45 to 60 seconds, typically less than 30 seconds, for example for 10 seconds or for another period, when the apparatus 1 was fully functional, as explained above, i.e. when it is in normal operating mode.

Once the emergency flow has been determined, the control means 210 control the main and secondary flow control means 220, 221, typically the main and secondary valve means 2200, 2210, in order to supply the gas containing NO, i.e. the NO/N₂ gaseous mixture, to the emergency flow thus obtained, as explained above.

The NO supply apparatus 1 can thus continue to supply the mixture based on NO, i.e. the NO/N₂ mixture, but to the emergency flow thus determined, despite the absence of measurements of respiratory gas flow, i.e. despite the interruption of transmission of these flow measurements (loss of signal), or even in the case of failure of the main flow control means 220, such as the MFC, which makes it possible to continue to care for the patient by supplying him with the NO at the desired posology or close to this posology. This improves greatly the safety of treatment for the patient.

A gas administration installation 100 can be used to administer nitrogen monoxide (NO) by inhalation, i.e. the final mixture obtained NO/O₂/N₂, to the people, i.e. patients, suffering from acute pulmonary arterial hypertension, in particular in order to dilate their pulmonary vessels, and increase their oxygenation by improving the pulmonary gaseous exchanges, in particular in order to treat pulmonary arterial hypertension of the newborn, or PPHN, acute respiratory distress syndrome, or ARDS observed, mainly in adults, or pulmonary hypertensions (PH) in cardiac surgery in adults or children.

We claim:

1. Installation (100) for supply of a gaseous mixture containing NO to a patient, comprising:
an NO supply apparatus (1) which is supplied with a gas containing NO in a given initial proportion, and configured to supply the gas containing NO;
a respiratory circuit (20; 21) comprising an injection device (24) which is configured to mix gas containing NO coming from the NO supply apparatus (1) with a flow of respiratory gas containing O₂ conveyed by the respiratory circuit (20; 21), and obtain a combined gaseous mixture containing NO and oxygen; and
a flow sensor (25) which is configured to measure at least one respiratory gas flow within the respiratory circuit (20; 21), and provide at least one respiratory gas flow measurement,
and wherein the NO supply apparatus (1) comprises:
an internal gas circuit (200) to convey the gas containing NO;
means for controlling the main and secondary flows (220, 221) which are configured to control the flow of gas containing NO conveyed by the internal gas circuit (200);
processor (211) control means (210) which are configured to:
determine at least one set gas flow containing NO to be supplied to the injection device (24), from at least one respiratory gas flow measurement carried out and supplied by the flow sensor (25); and
control at least part of the flow control means (220, 221) in order to supply the gas containing NO to the set flow determined;
and storage means (212),
characterised in that:
the storage means (212) are configured to store successive set flows of gas containing NO which have been determined by the control means (210); and
in the case of interruption of transmission to the control means (210), of any measurement of respiratory gas flow by the flow sensor (25), the control means (210) are configured to control the said flow control means (220, 221) in order to supply the gas containing NO to an emergency flow obtained or calculated from one or a plurality of set gas flows containing NO, which have been determined by the control means (210) and stored by the storage means (212), before the said interruption of transmission.

2. Installation according to claim 1, characterised in that the control means (210) are configured to determine successive set gas flows containing NO from a plurality of successive respiratory gas flow measurements carried out by the flow sensor (25) and supplied to the said control means (210).

3. Installation according to claim 2, characterised in that the storage means (212) are configured to store successive set gas flows containing NO which have been determined by the control means (210).

4. Installation according to claim 1, characterised in that the he emergency flow is obtained or calculated from a plurality of set flows which have been determined by the control means (210) during a given period (dt), before the said interruption of transmission.

5. Installation according to claim 4, characterised in that the emergency flow is calculated by obtaining a mean of the set flows which have been stored throughout the given period (dt).

6. Installation according to claim 4, characterised in that the given period (dt) is 30 seconds or less, preferably 20 seconds or less, or more preferably 10 seconds or less.

7. Installation according to claim 1, characterised in that the control means (210) are configured to determine the said at least one set gas flow containing NO to be supplied to the injection device (24), from at least one respiratory gas flow measurement carried out and supplied by the flow sensor (25), of a set NO content corresponding to the final proportion of NO required in the combined gaseous mixture, and from the initial proportion of NO in the gas containing NO which supplies the NO supply apparatus (1).

8. Installation according to claim 1, characterised in that the NO supply apparatus (1) is supplied with a gas containing an initial proportion of NO of between 100 and 1500 ppmv, in particular a gaseous mixture formed by nitrogen and NO.

9. Installation according to claim 1, characterised in that the NO supply apparatus (1) comprises dose regulation means which are configured to allow a user to set or select the set NO content corresponding to the final proportion of NO required in the combined gaseous mixture.

10. Installation according to claim 1, characterised in that it comprises a medical ventilator (50), configured to supply the flow of respiratory gas containing O₂ to the said respiratory circuit (20; 21).

11. Installation according to claim 4, characterised in that the given period (dt) is fixed and stored by the storage means (212), and preferably the storage means are incorporated in the control means (212).

12. Installation according to claim 9, characterised in that the set NO content is between 1 and 80 ppmv, typically between 5 and 40 ppmv.

13. Installation according to claim 9, characterised in that the dose regulation means form part of a man-machine interface (HMI) or user graphics interface (UGI).

14. Installation according to claim 9, characterised in that the dose regulation means comprise one or more touch keys which can be actuated by the user, displayed on a digital screen with a touch panel.

15. Installation according to claim 1, characterised in that the control means (212) comprise at least one (micro)processor arranged on at least one electronic board.
